SEQUENCE LISTING (2 sequences)

SEQ ID NO: 1 - protein, 293 aa, homo sapiens [sequence omitted]

SEQ ID NO: 2 - protein, 159 aa, homo sapiens
GHVGLGVKCSKEVATAIRGAIILAKLSIVPVRRGYWGNKIGKPHTVPCKVTGRCGSVLVRLIPAPRGTGIVSAPVPKKLLMMAGIDDCYTSARGCTATLGNFAKATFDAISKTYSYLTPDLWKETVFTKSPYQEFTDHLVKTHTRVSVQRTQAPAVATT

(19) United States
(12) Patent Application Publication
(10) Pub. No.: US 2024/0016893 A1
(43) Pub. Date: Jan. 18, 2024

(54) COMPOSITIONS AND METHODS FOR TREATING WOUNDS

(71) Applicant: IMAGINE PHARMA LLC, PITTSBURGH, PA (US)

(72) Inventors: NGOC THAI, PITTSBURGH, PA (US); JONATHAN POLLETT, PITTSBURGH, PA (US)

(21) Appl. No.: 18/028,104

(22) PCT Filed: Dec. 7, 2021
(86) PCT No.: PCT/US2021/062281

§ 371 Date: Mar. 23, 2023

Related U.S. Application Data
(60) Provisional application No. 63/122,746, filed on Dec. 8, 2020.

(51) Int. Cl. A61K 38/18; A61P 17/02; A61K 47/38

(52) U.S. Cl. CPC A61K 38/1858; A61P 17/02; A61K 47/38; A61K 38/1841

(57) ABSTRACT

Disclosed herein is a topical pharmaceutical composition for promoting wound healing, comprising: a therapeutically effective amount of a polypeptide of SEQ ID NO. 1 or 2, or a derivative or analog thereof, and a pharmaceutically acceptable carrier.

### CROSS REFERENCE TO RELATED APPLICATION

This application claims priority to U.S. provisional patent application No. 63/122,746, filed on Dec. 8, 2020, the contents of which is herein incorporated by reference in its entirety.

### INCORPORATION BY REFERENCE OF SEQUENCE LISTING

Herein is incorporated by reference a Sequence Listing. The file name is IMG105 NP US Replacement_ST25; the date of creation is Sep. 2, 2023; the size of the file is 4170 bytes.

### BACKGROUND

Wound healing is a dynamic process involving many factors and cell types including blood cells, fibroblasts, endothelial cells, and extracellular matrix including collagen and elastin. Normal wound healing is divided into several sequential phases that overlap in space and time: homeostasis, inflammation, granulation tissue formation, and tissue remodeling. Cutaneous injury elicits a complex wound healing process, which is an orchestration of cells, matrix components, and signaling factors that re-establishes the barrier function of skin. This phenomenon is characterized by an attenuated inflammatory response, differential expression of signaling factors, and regeneration of normal skin architecture. Several proteins such as Collagen and Elastin have been shown to play crucial roles in wound healing.

Collagen helps the body heal itself by preparing the wound bed, balancing wound chemistry, causing cell migration and growth, inducing granulation tissue, and improving overall skin strength. The role of collagen in these various chemicals, mechanical and biological factors, forms an environment conducive to wound healing, and ultimately, to wound closure. During injury/wound repair collagen binds to fibronectin and specific receptor sites of platelet membranes that cause platelet adhesion, aggregation, therefore releasing substances to initiate hemostasis. Furthermore, it acts as a chemotactic to monocytes and leukocytes and promotes autolysis in wound healing by using the body's enzymes and moisture to rehydrate, liquify devitalized tissues. Furthermore, collagen provides support for the growth of new capillaries and directly supports the growth, attachment, differentiation, and migration of keratinocytes to the damaged areas. The addition of collagen to injured animals has been shown to accelerate the wound healing process and thus represents a therapeutic potential product that may be beneficial in wound clinics in the future.

Elastin endows a range of mechanical and cell interactive properties to the skin. In adult wound healing, elastin is severely lacking and only a disorganized elastic fiber network is present after scar formation. The inherent properties of elastin make it a desirable inclusion to adult wound healing. Elastin imparts recoil and resistance and induces a range of cell activities, including cell migration and proliferation, matrix synthesis, and protease production.

Chronic wounds develop as a result of defective regulation of one or more of the complex molecular and biological events involved in proper healing. Chronic wounds in diabetics are one of the most common complications that affects millions of patients per year in the United States and costs the healthcare system billions of dollars for treatment options, which are often inadequate. Chronic wounds, especially diabetic foot ulcers, come with very high costs for the people suffering from it, with 25 billion dollars spent annually on treatment. Even though chronic wounds are not an uncommon problem and 9-12 million people suffer from them, there is a limited amount of wound care supplies, which results in an increased number of amputations, costing the healthcare system and the patient even more money. Wound healing and treatment continue to represent a major health challenge and consume a large amount of healthcare resources to improve patient's quality of life.

### SUMMARY

A composition for, and method of, treating a wound in a subject in need of such treatment is provided. A composition herein comprises a polypeptide according to SEQ ID NO. 1 or 2, or a derivative or analog thereof, in a vehicle suitable for transdermal delivery of the polypeptide. The method includes administering to the subject the composition comprising a polypeptide according to SEQ ID NO. 1 or 2, or a derivative or analog thereof, in a vehicle suitable for transdermal delivery of the polypeptide to a wound site. A wound site refers to a chronic wound, an abrasion, cut, burn, or site of a surgical procedure such as a skin graft. In one embodiment the polypeptide has at least 50%, 55%, 60%, 65%, 70%, 75%, 80%, 85%, 90%, 95%, 98% or 99% sequence identity to the amino acid sequence set forth in SEQ ID NO. 1 or 2.

The compositions herein can be administered to wounded skin and/or a wound topical cavity of a patient as a prophylactic or therapeutic dose, or as a cosmetic for skin, and may optionally be delivered by means of a wetted dressing.

### DETAILED DESCRIPTION OF THE DRAWINGS

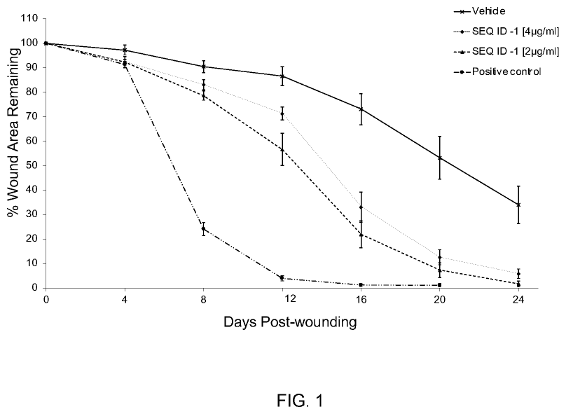

FIG. 1 shows that treatment with a topical composition comprising SEQ ID 1 was found to have a positive impact on wound closure relative to vehicle control treatment as measured by the percentage of wound area remaining at 4-, 8-, 12-, 16-, 20- and 24-days post wounding in the db/db diabetic impaired healing animal model.

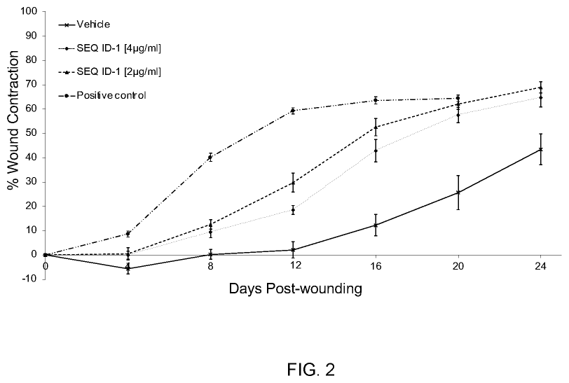

FIG. 2 shows that a topical composition comprising SEQ ID 1 was found to have a positive impact on wound contraction relative to vehicle control treatment as measured by the percentage of wound contraction at 4-, 8-, 12-, 16-, 20- and 24-days post wounding in the db/db diabetic impaired healing animal model.

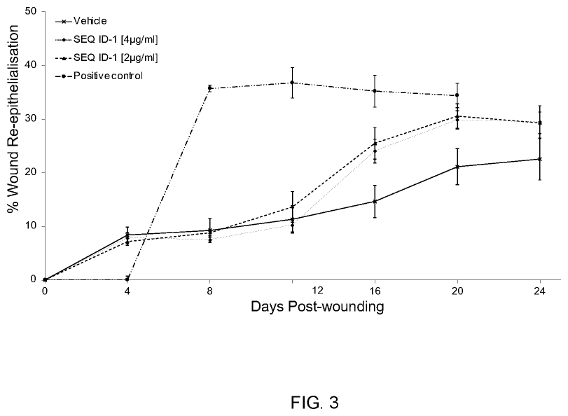

FIG. 3 shows that a topical composition comprising SEQ ID 1 was found to have a positive impact on wound re-epithelialization relative to vehicle control treatment as measured by the percentage of wound re-epithelialization at 4-, 8-, 12-, 16-, 20- and 24-days post wounding in the db/db diabetic impaired healing animal model.

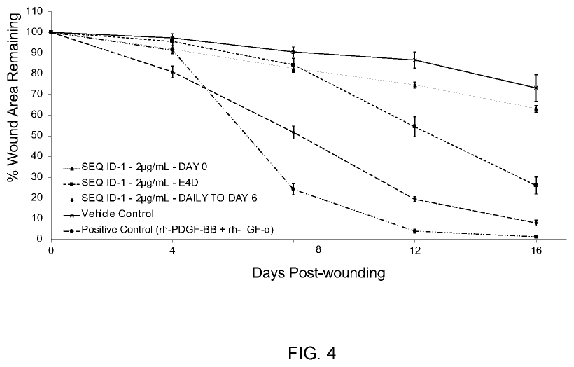

FIG. 4 shows that a topical composition comprising SEQ ID 1 (in differing dosage regimens) was found to have a positive impact on wound closure relative to vehicle control treatment as measured by the percentage of wound area remaining at 4-, 8-, 12-, and 16-days post wounding in the db/db diabetic impaired healing animal model.

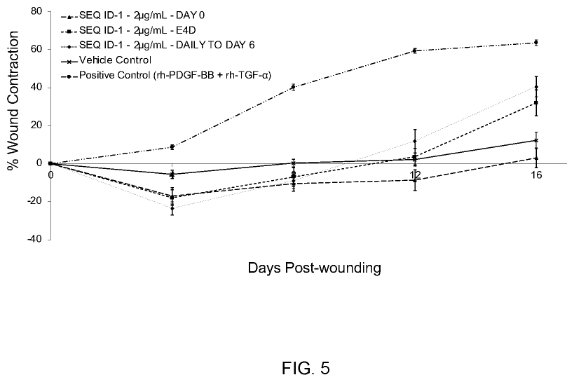

FIG. 5 shows that a topical composition comprising SEQ ID 1 (in differing dosage regimens) was found to have a positive impact on wound contraction relative to vehicle control treatment as measured by the percentage of wound contraction at 4-, 8-, 12-, and 16-days post wounding in the db/db diabetic impaired healing animal model.

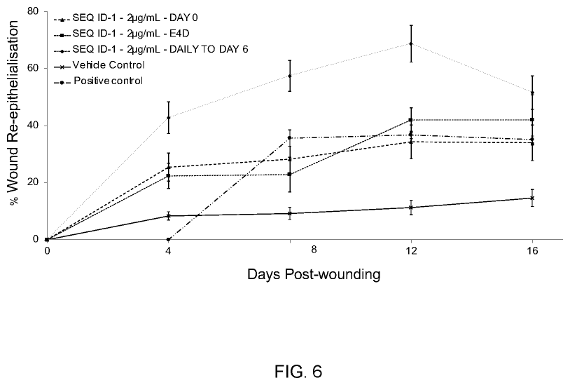

FIG. 6 shows that a topical composition comprising SEQ ID 1 (in differing dosage regimens) was found to have a positive impact on wound re-epithelialization relative to vehicle control treatment as measured by the percentage of wound re-epithelization at 4-, 8-, 12-, and 16-days post wounding in the db/db diabetic impaired healing animal model.

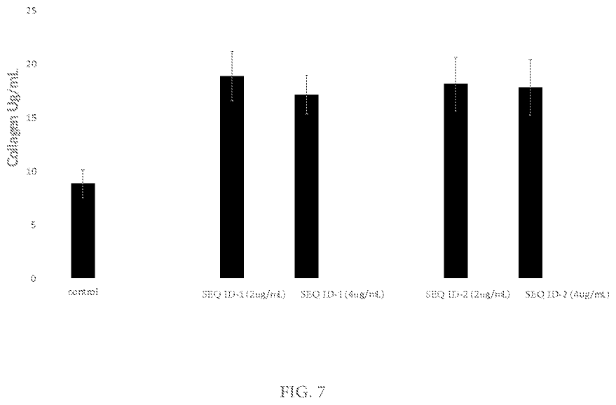

FIG. 7 shows that a composition comprising SEQ ID 1 or 2 facilitates wound healing by increasing the production of collagen by keratinocytes.

### DETAILED DESCRIPTION

The following detailed description is provided to aid those skilled in the art in practicing the compositions and methods disclosed herein, but should not be construed to limit, as modifications and variations in the embodiments disclosed herein may be made by those of ordinary skill in the art without departing from the scope and spirit of the present disclosure. All publications and other references cited in this application are hereby incorporated by reference in their entirety. In the event that subject matter incorporated by reference conflicts with subject matter disclosed herein, the subject matter of the present disclosure controls.

The following terms are used in this disclosure to describe different aspects of the compositions and methods disclosed herein. These terms are used for explanation purposes only.

As used herein “effective amount” refers to that amount of active ingredient, which, when administered to a subject is effective to promote healing and wound closure. In one embodiment, an effective amount of a topical composition comprising SEQ ID NO 1 or 2 is an amount in the range of about 0.1 μg/mL up to about 10 μg/mL and all values in between. Alternatively, an effective amount of topical composition comprising SEQ ID NO 1 or 2 is an amount in the range of about 0.01% w/v to about 10% w/v, and all values in between.

As used herein “formulation” or “composition” refer (interchangeably) to a solution, cream, ointment, paste, lotion, ointment, foam, spray, transdermal patch, or gel containing an effective amount of active ingredient, which is prepared so that it is suitable for administration to a wound site. If needed, the formulation may contain pharmaceutically acceptable carriers, excipients and/or one or more additives. Suitable additives are, for example: viscosity agents, antioxidants (e.g. ascorbic acid, methionine), coloring agents, preservatives, stabilizers, buffering agents, chelating agents (e.g. EDTA), binders, disinfecting agents, moisturizing agents, hyaluronic acid, antibacterial agents, anti-inflammatory and/or antifungal agents. The formulations disclosed herein may contain other active ingredient(s) in combination with the active ingredients described herein.

As used herein in the Examples, “IMG-1T” refers to a composition comprising a polypeptide according to SEQ. ID NO. 1 or 2 (the “active ingredient”).

As used herein SEQ. ID NO 1 refers to a 293 amino acid polypeptide comprising the following sequence:

MADDAGAAGGPGGPGGPGMGNRGGFRGGFGSGIRGRGRGRGRGRGRGRG
ARGGKAEDKEWMPVTKLGRLVKDMKIKSLEEIYLFSLPIKESEIIDFFL
GASLKDEVLKIMPVQKQTRAGQRTRFKAFVAIGDYNGHVGLGVKCSKEV
ATAIRGAIILAKLSIVPVRRGYWGNKIGKPHTVPCKVTGRCGSVLVRLI
PAPRGTGIVSAPVPKKLLMMAGIDDCYTSARGCTATLGNFAKATFDAIS
KTYSYLTPDLWKETVFTKSPYQEFTDHLVKTHTRVSVQRTQAPAVATT

As used herein SEQ ID NO 2 refers to a 159 amino acid polypeptide comprising the following sequence:

GHVGLGVKCSKEVATAIRGAIILAKLSIVPVRRGYWGNKIGKPHTVPCK
VTGRCGSVLVRLIPAPRGTGIVSAPVPKKLLMMAGIDDCYTSARGCTAT
LGNFAKATFDAISKTYSYLTPDLWKETVFTKSPYQEFTDHLVKTHTRVS
VQRTQAPAVATT

Contemplated herein are pharmaceutically acceptable salt forms of SEQ. ID NO. 1 or 2. Contemplated herein are derivatives of SEQ. ID NO. 1 or 2. Generally, a derivative of SEQ. ID NO. 1 or 2 comprises, for example, a fragment, one or more conservative amino acid substitutions, a chemically modified amino acid, and the like. Fragments of SEQ. ID NO. 1 include, for example, ¹M-¹³⁴N, ¹³⁵G-²⁰¹D, ¹³⁵G-²⁹³T, ¹⁶¹S-²³⁵N, and the like. A conservative amino acid substitution contemplated herein include, for example replacing, e.g., serine (S) with threonine (T), cysteine (C) with serine (S), lysine (K) with arginine, among others. See, e.g., Table 1 of WO9957141A1. Derivatives of SEQ. ID NO. 1, include, for example, acylated (e.g., RC(O)—, where R may be a C₁-C₁₆ alkyl, such as methyl) lysinyl moieties (e.g., ⁵⁴K, ⁵⁸K, ⁶⁵K, ⁷¹K, ²⁶³K, ²⁷⁵K, and the like), phosphorylated serinyl or tyrosinyl moieties (e.g., ²⁶⁴S, ²⁷⁰T, ²⁸¹S, and the like).

As used herein, “subject” or “individual” or “animal” or “patient” or “mammal” refers to any subject, in particular a mammalian subject, for which a diagnosis, prognosis or therapy is desired, for example, to a person.

As used herein, the terms “treat,” “treating” or “treatment,” and other grammatical equivalents as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disorder, or to a patient reporting one or more of the physiological symptoms, even though a diagnosis may not have been made.

As used herein, a “therapeutically effective amount”, is an amount of active ingredient capable of achieving a clinically relevant endpoint in a patient or patient population.

As used herein, a “wound” is intended to mean an injury or breakdown in the protective function of one or more layers of the skin; the loss of continuity of epithelium, with or without loss of underlying connective tissue (i.e. muscle, bone, nerves) following injury to the skin or underlying tissues/organs caused one or more factors, including: laceration, cut, abrasion, surgical procedure, burn (including chemicals), friction/shear force, pressure, or as a result of disease, such as from diabetic ulcers or carcinomas.

### Examples

The following information illustrates aspects of the pharmaceutical composition disclosed herein and its use for the treatment of a wound in a mammal (e.g., human) and should not be considered limiting on the subject matter claimed herein.

### Example 1

Patients with diabetes are prone to impaired wound healing, with foot ulceration being particularly prevalent. Delayed wound healing is also observed in certain diabetic animals, including the spontaneously diabetic (db/db) mouse (i.e., BKS.Cg-m Dock^{7m}+/+Lepr^{db}/J mice).

This exemplary study examined the effect of two concentrations of IMG-1T (in 0.5% HPMC), applied topically on the repair of full-thickness excisional skin wounds in this healing impaired model. The healing of wounds in receipt of IMG-1T (2.0 and 4.0 μg/mL) in 0.5% HPMC was examined and compared to that of similar wounds exposed to control (0.5% HPMC) treatment. Previous work by others this in vivo model, has clearly demonstrated enhanced wound healing following the topical application of a variety of recombinant human peptide growth factors; noticeable synergism being observed with certain growth factor combinations (See Brown et al. 1994, J. of Surgical Research, 56: 562-570). That being the case, wound closure data generated during this study was compared to historical positive control data from wounds treated with a combination of recombinant human platelet-derived growth factor-BB (rh-PDGF-BB) in combination with recombinant human Transforming Growth Factor-alpha (rh-TGF-alpha).

The impact of treatment was studied at both the macroscopic and histological levels. At the macroscopic level treatments were investigated in terms of (i) initiation of neo-dermal repair responses, and (ii) wound closure. Initiation of neo-dermal tissue formation was expressed in terms of the number (proportion) of wounds responding in each group at each time point. Wound closure was considered in terms of “overall reduction in open wound area remaining with time”, and its components wound contraction and wound re-epithelialization. At the histological level, wound tissues harvested on day 24 were investigated and compared in terms of:—i) granulation tissue deposition (depth), ii) % re-epithelialization; iii) collagen deposition, and iv) cellular proliferation.

The IMG-1T formulations (ie, topical compositions comprising SEQ ID NO 1 or 2) evaluated in this study were found to have a positive impact on the healing of wounds in the db/db diabetic mouse impaired healing model. IMG-1T was found to promote overall wound closure and both of its components (contraction and re-epithelialization), and was found to promote collagen deposition within newly formed granulation tissue.

Improved wound closure was observed with 2 μg/mL compared to 4 μg/mL, and there was a tendency towards improved outcomes with IMG-1T at 2 μg/mL in all other parameters assessed.

Materials:

IMG-1T formulation 1—HIGH—8 μg/mL in 1% HPMC (Sigma H7509)—diluted in sterile water (Ph Eur) to 4 μg/mL in 0.5% HPMC; IMG-1T formulation 2—LOW—4 μg/mL in 1% HPMC (Sigma H7509)—diluted in sterile water (Ph Eur) to 2 μg/mL in 0.5% HPMC; IMG-1T, 0.2 μm filter sterilized; Vehicle—0.5% HPMC (Sigma H7509); Historical Positive Control (Data)—Recombinant Human Platelet-derived Growth Factor-BB [rh-PDGF-BB] (Peprotec EC Ltd; 100-14B)+recombinant human Transforming Growth Factor-alpha [rh-TGF-alpha] (Peprotec EC Ltd: 100-16A) in 0.25% HPMC (Sigma H7509). Wounds received 100 μL per day (days 0 to 6).

The BKS. Cg-m Dock^{7m}+/+Lepr^{db}/J Diabetic (Impaired Healing) Mouse Model male diabetic mice (BKS.Cg-m Dock^{7m}+/+Lepr^{db}/J, Jackson Labs, Bar Harbour, ME, USA) aged approximately 8-9 weeks and were allowed to acclimate for one week prior to the start of the study. Animals were maintained according to proper regulations and specific requirements of diabetic animals. On day 0, mice were randomly allocated to one of 3 treatment regimens (groups 1 to 3 as described in Table 1. below).

TABLE 1
Experimental Groups

| | Treatment | | Treatment | | |
|---|---|---|---|---|---|
| Tx | (TFD = Tegaderm Film | | Application | Animal Codes & | |
| Group | Dressing) | Group name | (day) | harvesting | “n” |
| 1 | Vehicle Control - 0.5% HPMC | Control | 0, 4, 8, 12, 16 | IMG-01.01 to | 10 |
| | (+TFD) | | | 01.10 | |
| 2 | IMG-1T - 4 μg/mL in 0.5% | IMG-1TA [conc] | 0, 4, 8, 12, 16 | IMG-01.11 & | 10 |
| | HPMC + TFD | | | 01.20 | |
| 3 | IMG-1T - 2 μg/mL in 0.5% | IMG-1TB [conc] | 0, 4, 8, 12, 16 | IMG-01.21 & | 10 |
| | HPMC + TFD | | | 01.30 | |
| 4* | rh-PDGF-BB [10 μg] + rh-TGF- | Positive control | 0, 1, 2, 3, 4, 5 | HC.01 to HC.10 | 10 |
| | alpha [1 μg] - in 0.5% HMPC | | & 6) | | |
| | (100 μL) + Film Dressing | | | | |

Briefly, mice were anaesthetized using isoflurane and air, and their dorsal flank skin was clipped and cleansed according to protocol. A single standardized full-thickness wound (10 mm×10 mm) was created on the left dorsal flank approximately from the spine. Wounds were photographed with an identification plate and calibration rule and were then dressed with a piece of the transparent film dressing Tegaderm® Film (3M Deutschland GmbH, Germany). The materials under test (including the vehicle control) were then applied directly to the wound surface by injection through the film dressing using a 30G hypodermic needle (dose volume 100 μL). On post-wounding days 4, 8, 12, 16 & 20 all animals were re-anaesthetized, their film dressings and any free debris removed, and their wounds (and marginal skin) were gently cleaned using sterile saline-soaked gauze. Wounds were then assessed and digitally photographed (together with a calibration/identity plate). Tegaderm Film dressings were re-applied to all wounds and test materials/vehicle injected into the wound void (as on Day 0). Animals were recovered under warmed conditions after each anesthetic episode.

On post-wounding day 24, film dressings were removed and wounds cleaned, assessed and digitally photographed. After wound photography, blood samples were taken by cardiac puncture after which animals were terminated. Blood samples were taken into dipotassium EDTA tubes and centrifuged at 4° C. to generate plasma and was stored at −80° C. One hour prior to termination all animals received an i.p. injection (30 μg/g) of 5-bromo-2′-deoxyuridine (Sigma B5002) in normal saline to facilitate detection of cellular proliferation in histological sections. Wound and surrounding marginal tissue was then harvested from all wounds. Tissues were fixed (Neutral Buffered Formalin, Sigma) and embedded in paraffin wax to facilitate histological investigation.

Image Pro Plus image analysis software (version 4.1.0.0, Media Cybernetics, USA) was used to calculate wound closure from scaled wound images taken at each assessment point. As the process of wound closure results from the combined effects of wound contraction (the inward movement of marginal tissue) and re-epithelialization (wound resurfacing by the inward the migration of epithelial cells), wound closure over time was also considered with respect to these components.

The following assessments were made: percentage wound area remaining with time i.e., the open wound area remaining at a given time point, relative to the area of the same wound immediately after injury on day 0; percentage wound contraction with time i.e., the difference between the contracted wound area at a given time point and the original wound area, as a percentage of the original wound area; percentage re-epithelialization with time i.e., the contracted wound area at a given time minus the open wound area at that given time as a % of original wound area.

All wounds in the study were visually assessed on a daily basis until day 8—and subsequently on alternate days until day 20—to establish their “healing” status. Each wound was scored as to whether it was displaying “neo-dermal tissue generation activity” within the central wound area. Scoring was undertaken independently by two independent observers and the average % of wounds displaying “neo-dermal tissue generation activity” was compared between treatment groups at each assessment point. Neo-dermal tissue formation was considered to have started when blood vessels within the fascia of the wound base were concealed by overlying “material”. This concealment may result from the formation of cloudy exudate, polymerized/semi-polymerized fibrin or granulation tissue. Invariably, the first sign of neo-dermal tissue initiation is the formation of a reddish exudate within the wound void.

On post-wounding day 24, all animals were painlessly killed by compliant method (exsanguination confirmed by cervical dislocation). Wound and surrounding normal tissue was excised, fixed in 10% buffered formalin (Sigma, UK), processed and embedded in paraffin wax. One hour prior to termination all animals received an i.p. injection (30 μg/g) of 5-bromo-2′-deoxyuridine (Sigma B5002) to facilitate assessment of cellular proliferation. Excised tissue was sandwiched between two pieces of foam, prior to being placed in fixative, to reduce the extent of tissue curling. Fixed specimens were trimmed and bisected in a cranio-caudal direction, generating two half wounds per site. Both halves were processed and embedded in paraffin wax. Specimens were orientated in such a fashion as to ensure that appropriate transverse sections of the wound could be taken. Embedded wounds were then sectioned (6 μm) and stained with Haematoxylin & Eosin, and stained sections were digitally scanned.

Sections were stained and evaluated as described below:
1. Hematoxylin and Eosin (H&E)—to facilitate quantification of granulation tissue formation and % re-epithelialization.
2. Anti-BrdU antibody—to visualize and quantify proliferating cells within granulation tissue
3. Picrosirius red—to visualize collagenous structures and quantify collagen deposition.

Measurement of Granulation Tissue Deposition (in Terms of Granulation Tissue Depth, or GTD)

Granulation tissue deposition was measured in terms of Granulation. GTD was measured at 9 different sites across the width of each wound using Aperio ImageScope image analysis software (Leica Biosystems, UK). These measurements were then averaged to give a single granulation tissue depth measurement for each wound under study. Based on the cross-section of the wound area, the GTD distance (d) of the 9 different sites included three left-hand epithelialized sites (A), three non-epithelialized sites (C), and three right-hand epithelialized sites (B). The % re-epithelialization was calculated from the sum of the average distances for the epithelialized sites (A+B) divided by the sum of the average distances for the epithelialized sites and the non-epithelialized sites (A+B+C), where the ratio was multiplied by 100.

Measurement of Wound Re-Epithelialization

The extent of re-epithelialization from the left and right margins of each wound was measured from H&E-stained sections using Aperio ImageScope image analysis software (Leica Biosystems, UK). Wound re-epithelialization was expressed as the percentage of the wound surface epithelialized.

Proliferating Cells within Wounds

Proliferating cells within the neo-dermal and neo-epidermal compartments of wounds were specifically detected using BrdU:anti-BrdU immunostaining as described by Kitano et al. 2001. One hour prior to termination all animals received an i.p. injection (30 μg/g) of 5-bromo-2′-deoxyuridine (BrdU, Sigma). BrdU uptake by proliferating cells was then detected in histological sections by immunostaining for BrdU—in tandem with standard ABComplex immunoperoxidase detection techniques. The number of proliferating cells was counted in three wound regions (outer, intermediate and central regions). For each region of each wound, two areas of interest (each 200×200 μm) were selected and the number of proliferating cells determined using QuPath software. The average number of proliferating cells was calculated for each wound as a whole and for each of three wound regions. These whole wound and regional averages were compared between treatment groups (using appropriate statistical analysis techniques).

Collagen Deposition

Sections were stained with picrosirius red (PSR) in order to visualize the deposition of collagen within newly formed granulation tissue. Collagen deposition was measured in three wound regions (outer, intermediate and central regions). For each region of each wound, two areas of interest (each 200×200 μm) were selected and the percentage area ‘occupied’ by PSR stained collagen was determined. The average percentage area of collagen staining was calculated for each wound as a whole and for each of three wound regions. These whole wound and regional averages were compared between treatment groups (using appropriate statistical analysis techniques).

Wound Area Remaining

Each wound was digitally photographed, along with an identification/calibration plate, immediately after injury and subsequently on days 4, 8, 12, 16, 20 and 24. For a given wound at a given time point, wound closure was expressed as the percentage wound area remaining—relative to the initial wound area immediately after injury (i.e., day 0). Mean percentage wound area remaining data for all treatment groups are described in Table 2 below.

TABLE 2
Percentage “Wound Area Remaining” data for all study groups.
% Wound area remaining with time - open wound area (mean +/− standard error)

| | | Days post wounding: | | | | | |
|---|---|---|---|---|---|---|---|
| Group/Treatment | | 4 | 8 | 12 | 16 | 20 | 24 |
| 1 | Vehicle | 97.28 ± 2.03 | 90.51 ± 2.43 | 86.60 ± 3.87 | 73.13 ± 6.36 | 53.26 ± 8.72 | 34.07 ± 7.64 |
| 2 | IMG-1T [4 | 92.29 ± 1.15 | 83.12 ± 2.06 | 71.34 ± 2.65 | 33.12 ± 6.17 | 12.63 ± 3.08 | 5.91 ± 1.93 |
| | μg/mL] | | | | | | |
| 3 | IMG-1T [2 | 92.36 ± 2.28 | 78.68 ± 1.88 | 56.66 ± 6.56 | 21.97 ± 5.46 | 7.36 ± 2.97 | 1.79 ± 1.13 |
| | μg/mL] | | | | | | |
| 4 | Positive Control | 91.38 ± 1.21 | 24.16 ± 2.66 | 4.00 ± 1.04 | 1.29 ± 0.57 | 1.20 ± 0.73 | 1.79 ± 1.13 |

According to the data (Table 2—above):

Wound closure profiles of “% wound area remaining with time” data, were found to differ between treatment groups (see FIG. 1). The greatest level of wound closure was observed in the Positive Control treatment group, the lowest level was observed with the Vehicle Control group—and the IMG-1T treatment groups demonstrated increased levels of wound closure relative to the Vehicle Control and reduced levels relative to the Positive Control. Wounds in receipt of the Positive Control demonstrated significantly increased wound closure relative to the Vehicle Control group from day 4 onwards (p≤0.035), and relative to the two IMG-1T—treated groups from day 8 onwards (p≤0.023). Wounds in receipt of IMG-1T [4 μg/mL] demonstrated significantly increased wound closure relative to the Vehicle Control group from day 8 onwards (p≤0.023). Wounds in receipt of IMG-1T [2 μg/mL] demonstrated significantly increased wound closure relative to the Vehicle Control group from day 8 onwards (p≤0.002). On comparison of the two IMG-1T treatment groups; increased wound closure was observed with 2 μg/mL compared to 4 μg/mL. This was found to be statistically significant on day 12 only (p=0.035).

Contraction is the centripetal movement of the wound margins—due to the compaction of granulation tissue within the “body” of the wound. The “compactional” forces, that drive this process, are thought to reside in cells of the fibroblast lineage. In this study, % contraction was calculated as:

$$\% contraction = \frac{\begin{matrix}The area defined by the boundary of normal dermis and the \\ “ repairing neo - dermis ”\end{matrix}}{The original wound area ( day 0 )} \times 100$$

Mean percentage wound contraction data for all treatment groups are described in table 3 (below).

TABLE 3
Summary of “percentage wound contraction” data. % Wound contraction
with time - open wound area (mean +/− standard error)

| Group/ | | Days post wounding: | | | | | |
|---|---|---|---|---|---|---|---|
| Treatment | | 4 | 8 | 12 | 16 | 20 | 24 |
| 1 | Vehicle | −5.6 ± 2.2 | 0.3 ± 2.0 | 2.1 ± 3.4 | 12.3 ± 4.4 | 25.7 ± 7.0 | 43.4 ± 6.3 |
| 2 | IMG-1T [4 | −0.1 ± 1.6 | 9.4 ± 2.3 | 18.5 ± 1.9 | 42.9 ± 4.6 | 57.6 ± 3.2 | 64.7 ± 3.8 |
| | μg/mL] | | | | | | |
| 3 | IMG-1T [2 | 0.5 ± 2.5 | 12.5 ± 1.9 | 29.7 ± 3.9 | 52.6 ± 3.6 | 62.1 ± 2.3 | 68.9 ± 2.3 |
| | μg/mL] | | | | | | |
| 4 | Positive Control | 8.62 ± 1.21 | 40.19 ± 1.68 | 59.26 ± 1.26 | 63.55 ± 1.48 | 64.43 ± 1.31 | |

According to the data (Table 3—above), graphically displayed below in FIG. 2, the following points are apparent: Wound closure profiles of “% wound contraction” data, were found to differ noticeably between treatment groups, with the lowest levels of contraction observed in the Vehicle Control group (see FIG. 2). Wounds in receipt of the Positive Control (historical data) demonstrated significantly increased wound contraction relative to the Vehicle Control group from day 4 onwards (p≤0.002), and significantly increased wound contraction relative to IMG-1T treated wounds from day 4 to day 16 (p≤0.019) with similar levels of contraction observed on day 20 (FIG. 2). Wounds in receipt of IMG-1T [4 μg/mL] demonstrated significantly increased wound contraction relative to the Vehicle Control group from day 8 onwards (p≤0.007), with near-significance on day 4 (p=0.075). Wounds in receipt of IMG-1T [2 μg/mL] demonstrated significantly increased wound contraction relative to the Vehicle Control group from day 8 onwards (p≤0.002). On comparison of the two IMG-1T treatment groups; increased wound contraction was observed with 2 μg/mL compared to 4 μg/mL. This was found to be statistically significant on day 12 only (p=0.003).

For a given wound, at a given time point, the area of re-epithelialization was expressed as a percentage of the original area of that wound immediately after injury. Mean percentage wound re-epithelialization data for all treatment groups are described in table 4 (below).

TABLE 4
Summary of “percentage wound re-epithelialization” data % Wound re-
epithelialization with time (mean +/− standard error)

| Group/ | | Days post wounding: | | | | | |
|---|---|---|---|---|---|---|---|
| Treatment | | 4 | 8 | 12 | 16 | 20 | 24 |
| 1 | Vehicle | 8.4 ± 1.5 | 9.2 ± 2.2 | 11.3 ± 2.5 | 14.6 ± 3.0 | 21.1 ± 3.4 | 22.5 ± 3.9 |
| 2 | IMG-1T [4 | 7.8 ± 1.0 | 7.4 ± 0.5 | 10.2 ± 1.3 | 24.0 ± 2.2 | 29.8 ± 1.7 | 29.4 ± 3.1 |
| | μg/mL] | | | | | | |
| 3 | IMG-1T [2 | 7.1 ± 0.7 | 8.8 ± 0.6 | 13.6 ± 2.9 | 25.5 ± 3.0 | 30.5 ± 2.3 | 29.3 ± 2.0 |
| | μg/mL] | | | | | | |
| 4 | Positive Control | 0.0 ± 0.0 | 35.7 ± 2.9 | 36.7 ± 1.3 | 35.2 ± 1.2 | 34.4 ± 1.2 | 1.79 ± 1.13 |

According to the data (table 4—above), graphically displayed in FIG. 3, the following points are apparent: re-epithelialization was first measurable on day 4 post-wounding in all groups with the exception of the positive control group; wound closure profiles of “% wound re-epithelialization” data, were found to differ noticeably between treatment groups (see FIG. 3); wounds in receipt of the Positive Control (historical data) were found to display: i. significantly reduced re-epithelialization than all other treatment groups on day 4 (p=0.000); ii. significantly increased wound re-epithelialization relative to the Vehicle Control group on days 8 to 20 (130.015); iii. significantly increased wound re-epithelialization relative to both IMG-treatment groups on days 8 to 16 (130.015). Wounds in receipt of IMG-1T [4 μg/mL] demonstrated significantly increased wound re-epithelialization relative to the Vehicle Control group on days 16 and 20 (p≤0.035).

Assessments were made from histological sections of each wound (harvested on day 24 post-wounding); namely measurements of: i) granulation tissue formation (depth), ii) % re-epithelialization, iii) number of proliferative cells, and iv) collagen deposition. Measurement of granulation tissue deposition (in terms of granulation tissue depth, GTD) measurements were made (using Aperio ImageScope image analysis software (Leica Biosystems, UK)) from digitally enlarged ×20 magnification photomicrographic scans of H&E sections of each experimental wound. These measurements were then averaged to give a single granulation tissue depth measurement for each wound.

According to the data, the following points are apparent: Vehicle Control treatment gave rise to the lowest average GTD (avg. 189.41 μm). Treatment with IMG-1T at 2 μg/mL and 4 μg/mL resulted in avg. GTDs of 237.64 μm and 235.41 μm, respectively. The differences in GTD observed between the two IMG-1T treatment regimens and that observed following Vehicle Control treatment did not prove to be statistically significant. No significant difference in GTD was observed between the two IMG-1T treatment regimens.

Assessments were made from histological sections of each wound on collagen deposition in the wounds. Based on the collagen deposition data (not shown), the following was apparent: a. Higher levels of collagen were observed in outer and intermediate regions compared to central regions for all treatment groups; b. Higher levels of collagen were deposited following IMG-1T treatment, compared to vehicle control treatment, in all wound regions, reaching significance in the outer and intermediate regions; c. There was a tendency towards higher collagen deposition with IMG-1T at 2 μg/mL compared to 4 μg/mL.

The IMG-1T formulations evaluated in this Example 1 were found to have a positive impact on the healing of wounds in the db/db diabetic mouse impaired healing model. IMG-1T was found to promote overall wound closure and its components contraction and re-epithelialization, and was found to promote collagen deposition within newly formed granulation tissue. Improved wound closure was observed with 2 μg/mL and 4 μg/mL.

### Example 2

This exemplary study examined the effect of three alternative dosing regimens of IMG-1T in 0.5% HPMC applied topically on the repair of full-thickness excisional skin wounds in the healing-impaired model described in Example 1. The healing of wounds in receipt of IMG-1T (2.0 μg/mL) in 0.5% HPMC applied on day 0 post-wounding only was examined and compared to that of similar wounds exposed to application every 4 days (i.e., days 0, 4, 8 & 12 post-wounding) and to those exposed to applications every day until day 6 post-wounding (7 applications in total). Wound closure data generated in this study was compared to vehicle control data (i.e., from wounds exposed to 0.5% HPMC on days 0, 4, 12 & 16) from Example 1.

As stated above, previous work using this same vivo model has clearly demonstrated enhanced wound healing following the topical application of a variety of recombinant human peptide growth factors (Brown et al.). That being the case, wound closure data generated during this study was compared to historical positive control data from wounds treated with a combination of recombinant human platelet-derived growth factor-BB (rh-PDGF-BB) in combination with recombinant human Transforming Growth Factor-alpha (rh-TGF-alpha).

The impact of treatment was studied at the macroscopic level in terms of: (i) initiation of neo-dermal repair responses, and (ii) wound closure. Initiation of neo-dermal tissue formation was expressed in terms of the number (proportion) of wounds responding in each group at each time point. Wound closure was considered in terms of “overall reduction in open wound area remaining with time”, and its components wound contraction and wound re-epithelialization.

In this study a single 100 μL dose of IMG-1T (2 μg/mL in 0.5% HPMC) applied on Day 0 was found to significantly increase the overall rate of wound closure compared to vehicle control treatment. On consideration of the components of wound healing, namely, wound contraction and wound re-epithelialization; this single dose resulted in significantly increased re-epithelialization, in tandem with reduced wound contraction.

Application of IMG-1T (100 μL, 2 μg/mL) every four days (E4D) resulted in significantly increased wound closure compared to i) vehicle control treatment (E4D) and ii) IMG-1T applied on ‘Day 0 only’. This observed increase in wound closure, compared to the vehicle control group, consisted of both significantly increased wound contraction and increased wound re-epithelialization—suggesting a positive impact of IMG-1T on both components of wound closure. Dosing with IMG-1T ‘E4D’ also significantly increased the proportion of wounds demonstrating initiation of wound healing.

Application of IMG-1T (100 μL, 2 μg/mL) ‘Daily to day 6’ resulted in significantly increased wound closure compared to i) vehicle control treatment (E4D), ii) IMG-1T on ‘Day 0 only’, and iii) IMG-1T applied E4D. This observed increase in wound closure, was again due to both increased contraction and re-epithelialization. On comparison with the historical positive control (also dosed ‘Daily to Day 6’)—overall closure was found to be lower following treatment with IMG-1T ‘Daily to Day 6’. This lower overall closure resulted from substantially lower contraction, in tandem a significant though less substantial elevation in re-epithelialization. The proportion of wounds demonstrating initiation of wound healing was found to be the same as with the positive control treatment.

Materials:

IMG-1T, 0.2 μm filter sterilized; IMG-1T—4 μg/mL in 1% HPMC (Sigma H7509). Diluted in sterile water (Ph Eur) to 2 μg/mL in 0.5% HPMC; Historical Vehicle Control (Data)—Example 1; 0.5% HPMC (Sigma H7509); Historical Positive Control (Data)—Recombinant Human Platelet-derived Growth Factor-BB [rh-PDGF-BB] (Peprotec EC Ltd; 100-14B)+recombinant human Transforming Growth Factor-alpha [rh-TGF-alpha] (Peprotec EC Ltd; 100-16A) in 0.25% HPMC (Sigma H7509). Wounds received 100 μL per day (days 0 to 6).

The materials and methods employed in this study are described in Example 1, including the BKS.Cg-m Dock^{7m}+/+Lepr^{db}/J Diabetic Mouse Model. On day 0, mice were randomly allocated to one of 3 treatment regimens (groups 1 to 3 as described in Table 5. below).

TABLE 5
Experimental Groups of Example 2

| | Treatment | | | | |
|---|---|---|---|---|---|
| Tx | (TFD = Tegaderm Film | | Treatment | Animal Codes & | |
| Group | Dressing) | Group name | Application (day) | harvesting | “n” |
| 1 | IMG-1T - 2 μg/mL in 0.5% | IMG-1T DO | 0 only | IMG-02.01 to | 10 |
| | HPMC (100 μL) + TFD | | | 02.10 | |
| 2 | IMG-1T - 2 μg/mL in 0.5% | IMG-1T - E4D | 0, 4, 8 & 12 | IMG-02.11 to | 10 |
| | HPMC (100 μL) + TFD | | | 02.20 | |
| 3 | IMG-1T - 2 μg/mL in 0.5% | IMG-1T - ED6 | 0, 1, 2, 3, 4, 5 & 6 | IMG-02.21 to | 10 |
| | HPMC (100 μL) + TFD | | | 02.30 | |
| 4 | Vehicle Control - 0.5% HPMC | Control | 0, 4, 8, 12, 16 | IMG-01.01 to | 10 |
| | (100 μL) + TFD | (historical) | | 01.10 | |
| 5 | rh-PDGF-BB [10 μg] + rh-TGF- | Positive control | 0, 1, 2, 3, 4, 5 & 6 | HC.01 to HC.10 | 10 |
| | alpha [1 μg] - in 0.5% HMPC | (historical) | | | |
| | (100 μL) + TFD | | | | |

Briefly, mice were anaesthetized using isoflurane and air, and their dorsal flank skin was clipped and cleansed according to protocol. A single standardized full-thickness wound (10 mm×10 mm) was created on the left dorsal flank approximately 5 mm from the spine. Wounds were photographed with an identification plate and calibration rule and were then dressed with a piece of the transparent film dressing Tegaderm Film (3M Deutschland GmbH, Germany). IMG-1T (2 μg/mL in 0.5% HPMC) was then be applied directly to the wound surface by injection through the film dressing using a 30G hypodermic needle (dose volume 100 μL). Animals in group 1 received IMG-1T on day 0 (immediately after wounding) only. Animals in group 2 received IMG-1T on days 0, 4, 8 and 12; while those in group 3 received IMG-1T on a daily basis from day 0 until post-wounding day 6 (7 applications in total). On post-wounding days 4, 8, 12 & 16 all animals were re-anaesthetized, their film dressings and any free debris removed, and their wounds (and marginal skin) were gently cleaned using sterile saline-soaked gauze. Wounds were then assessed and digitally photographed (together with a calibration/identity plate). Tegaderm® Film dressings were re-applied to all wounds and where applicable test materials injected into the wound void (as on Day 0). Animals were recovered under warmed conditions after each anesthetic episode.

On post-wounding day 16 film dressings were removed and wounds cleaned, assessed and digitally photographed. After wound photography, blood samples were taken by cardiac puncture after which animals were terminated. Wound and surrounding marginal tissue, and liver and kidney tissue samples, were then harvested from all animals. One hour prior to termination all animals received an i.p. injection (30 μg/g) of 5-bromo-2′-deoxyuridine (Sigma B5002) in normal saline to facilitate detection of cellular proliferation in histological sections.

Each wound was digitally photographed, along with an identification/calibration plate, immediately after injury and subsequently on days 4, 8, 12 and 16. For a given wound at a given time point, wound closure was expressed as the percentage wound area remaining—relative to the initial wound area immediately after injury (i.e., day 0). Mean percentage wound area remaining data for all treatment groups are described in table 6 below.

TABLE 6
Percentage “Wound Area Remaining” data for all study groups.
% Wound area remaining with time -_ open wound area (mean +/− standard error)

| Group/ | | Days post wounding: | | | |
|---|---|---|---|---|---|
| Treatment | | 4 | 8 | 12 | 16 |
| 1 | IMG-1T - 2 μg/mL DAY | 91.63 ± 1.97 | 82.36 ± 1.78 | 74.45 ± 1.44 | 63.01 ± 1.56 |
| | 0 only | | | | |
| 2 | IMG-1T - 2 μg/mL E4D | 95.60 ± 2.05 | 84.21 ± 3.26 | 54.39 ± 4.84 | 26.01 ± 4.07 |
| 3 | IMG-1T - 2 μg/mL Daily | 80.84 ± 2.83 | 51.60 ± 3.11 | 19.45 ± 1.15 | 7.93 ± 1.37 |
| | to D 6 | | | | |
| 4 | Vehicle Control - E4D | 97.28 ± 2.03 | 90.51 ± 2.43 | 86.60 ± 3.87 | 73.13 ± 6.36 |
| 5 | rh-PDGF-BB [10 μg] + | 91.38 ± 1.21 | 24.16 ± 2.66 | 4.00 ± 1.04 | 1.29 ± 0.57 |
| | rh-TGF-alpha [1 μg] | | | | |

The raw wound closure data and corresponding percentage wound area data generated during this study are displayed graphically in FIG. 4, and subsequent to statistical analysis of that data, the following points are apparent: Wound closure profiles of “% wound area remaining with time” data, were found to differ between treatment groups (see FIG. 4). The greatest level of wound closure was observed in the Positive Control treatment group, the lowest level was observed with the Vehicle Control group (historical data)—and the IMG-1T treatment groups demonstrated increased levels of wound closure relative to the Vehicle Control and reduced levels relative to the Positive Control. Wounds in receipt of the Positive Control (historical data) demonstrated significantly increased wound closure relative to: i. Vehicle Control treated wounds (historical data) from day 4 onwards (130.035); ii. IMG-1T ‘Day 0 only’ treated wounds from day 8 onwards (p=0.000); iii. IMG-1T ‘E4D’ treated wounds from day 8 onwards (p=0.000); iv. IMG-1T ‘Daily to Day 6’ treated wounds on days 12 & 16 (p=0.000). Wounds in receipt of IMG-1T applied ‘Day 0 only’—demonstrated significantly increased wound closure relative to the Vehicle Control treated wounds (historical data) on days 4 to 12 (130.035). Wounds in receipt of IMG-1T applied ‘E4D’—demonstrated significantly increased wound closure relative to Vehicle Control treated wounds (historical data) on days 12 & 16 (p=0.000). Wounds in receipt of IMG-1T applied ‘Daily to Day 6)—demonstrated significantly increased wound closure relative to the Vehicle Control treated wounds (historical data) from day 4 onwards (p=0.000). On comparison of the different dosing frequencies, the following observations were made: i. Application ‘E4D’ resulted in significantly increased closure compared to application on ‘Day 0 only’—on days 12 & 16 (p=0.000); ii. ‘Daily to Day 6’ application resulted in significantly increased closure compared to application on ‘Day 0 only’ from day 4 onwards (p≤0.015); iii. ‘Daily to Day 6’ application resulted in significantly increased closure compared to application ‘E4D’ from day 4 onwards (p≤0.015).

All three IMG-1T dosing regimens significantly increased wound closure compared to historical vehicle control data (E4D). On comparison of the three dosing regimens the greatest increase in wound closure was observed with ‘Daily to Day 6’ application—followed by ‘E4D’—followed by dosing on ‘Day 0 only’.

The raw wound contraction data and corresponding percentage wound contraction data generated during this study are tabulated in Table 7 and displayed graphically in FIG. 5:

TABLE 7
Summary of “percentage wound contraction” data: % Wound contraction
with time (mean +/− standard error)

| Group/ | | Days post wounding: | | | |
|---|---|---|---|---|---|
| Treatment | | 4 | 8 | 12 | 16 |
| 1 | IMG-1T - 2 μg/mL DAY | −17.08 ± 4.33 | −10.54 ± 4.00 | −8.75 ± 5.43 | 3.00 ± 5.11 |
| | 0 only | | | | |
| 2 | IMG-1T - 2 μg/mL E4D | −17.99 ± 4.18 | −7.04 ± 4.92 | 3.62 ± 4.35 | 31.97 ± 6.86 |
| 3 | IMG-1T - 2 μg/mL Daily | −23.62 ± 3.48 | −9.05 ± 4.07 | 11.82 ± 6.17 | 40.49 ± 5.29 |
| | to D 6 | | | | |
| 4 | Vehicle Control - E4D | −5.64 ± 2.21 | 0.26 ± 2.03 | 2.10 ± 3.35 | 12.25 ± 4.36 |
| 5 | rh-PDGF-BB [10 μg] + rh- | 8.62 ± 1.21 | 40.19 ± 1.68 | 59.26 ± 1.26 | 63.55 ± 1.48 |
| | TGF-alpha [1 μg] | | | | |

According to the data of Table 7, the following points are apparent (and as shown in FIG. 5): Wound closure profiles of “% wound contraction” data, were found to differ noticeably between treatment groups, with the highest levels of contraction observed in the Positive Control (historical data) group. Wounds in receipt of the Positive Control (historical data) demonstrated significantly increased wound contraction relative to all other treatment groups from day 4 onwards (p=0.000). Wounds in receipt of IMG-1T applied ‘Day 0 only’ demonstrated reduced wound contraction relative to Vehicle Control treated wounds (historical data). This was found to be statistically significant on day 8 (p=0.029), with near significance on day 4 (p=0.052). Wounds in receipt of IMG-1T ‘E4D’—demonstrated significantly reduced wound contraction relative to wounds treated with the Vehicle Control (historical data) on day 4 (p=0.029). This was followed by an increased rate of contraction by IMG-1T treated wounds—that resulted in significantly increased contraction compared to vehicle control treatment by day 16 (p=0.035). Wounds in receipt of IMG-1T applied ‘Daily to Day 6’ demonstrated significantly reduced wound contraction relative to wounds given the Vehicle Control (historical data) on day 4 (p=0.001) and near-significantly reduced contraction on day 8 (p=0.052). This was followed by an increased rate of contraction by IMG-1T treated wounds that resulted in significantly increased contraction compared to vehicle control treatment by day 16 (p=0.002). On comparison of the different dosing frequencies with IMG-1T, the following observations were made: i. ‘E4D’ application resulted in increased contraction compared to application on ‘Day 0 only’ from day 8 onwards, reaching statistical significance on day 16 only (p=0.002); ii. ‘Daily to Day 6’ application resulted in significantly increased contraction compared to application on ‘Day 0 only’ on days 12 & 16 (p≤0.035); iii. ‘Daily to Day 6’ application resulted in marginally increased contraction compared to application ‘E4D’ on days 12 & 16—no significant differences were noted.

For a given wound, at a given time point, the area of re-epithelialization was expressed as a percentage of the original area of that wound immediately after injury. Mean percentage wound re-epithelialization data for all treatment groups are described in table 8 below.

TABLE 8
Summary of “percentage wound re-epithelialization” data % Wound re-
epithelialization with time (mean +/− standard error)

| Group/ | | Days post wounding: | | | |
|---|---|---|---|---|---|
| Treatment | | 4 | 8 | 12 | 16 |
| 1 | IMG-1T - 2 μg/mL DAY 0 only | 25.4 ± 4.9 | 28.2 ± 4.6 | 34.3 ± 5.9 | 34.0 ± 6.2 |
| 2 | IMG-1T - 2 μg/mL E4D | 22.4 ± 4.4 | 22.8 ± 6.1 | 42.0 ± 4.3 | 42.0 ± 8.8 |
| 3 | IMG-1T - 2 μg/mL Daily to D 6 | 42.8 ± 5.5 | 57.5 ± 5.4 | 68.7 ± 6.4 | 51.6 ± 5.9 |
| 4 | Vehicle Control - E4D | 8.36 ± 1.46 | 9.23 ± 2.2 | 11.31 ± 2.5 | 14.61 ± 3.0 |
| 5 | rh-PDGF-BB [10 μg] + rh-TGF-alpha | 0.0 ± 0.0 | 35.7 ± 2.9 | 36.7 ± 1.3 | 35.2 ± 1.2 |
| | [1 μg] | | | | |

According to the data (table 8—above), graphically displayed in FIG. 6, the following points are apparent: Re-epithelialization was first measurable on day 4 post-wounding in all groups with the exception of the positive control group. Wound closure profiles of “(% wound re-epithelialization” data, were found to differ noticeably between treatment groups (See FIG. 6). Wounds in receipt of the Positive Control (historical data) were found to display: i. Significantly reduced re-epithelialization than all other treatment groups on day 4 (p=0.000); ii. Significantly increased wound re-epithelialization relative to Vehicle Control treated wounds on days 8 to 20 (p≤0.015); iii. Similar levels of re-epithelialization to that of wounds given IMG-1T on Day 0 only) from day 8 onwards; iv. Marginally increased re-epithelialization relative to wounds given IMG-1T ‘E4D’—on day 8 (p=0.089), with similar levels thereafter; v. Significantly reduced levels of re-epithelialization relative to wounds given IMG-1T ‘Daily to Day 6’ on days 8 to 16 (p<0.029). Wounds in receipt of IMG-1T on ‘Day 0 only’ displayed significantly increased wound re-epithelialization relative to Vehicle Control treated wounds at all time points (p≤0.035). Wounds in receipt of IMG-1T ‘E4D’ demonstrated significantly increased wound re-epithelialization relative to Vehicle Control treated wounds on days 4, 12 & 16 (p≤0.035) with near-significance on day 8 (p=0.052). Wounds in receipt of IMG-1T ‘Daily to Day 6’ displayed significantly increased wound re-epithelialization relative to Vehicle Control treated wounds at all time points (p=0.000).

On comparison of the different dosing frequencies with IMG-1T, the following observations were made: i. Application ‘E4D’ resulted in similar levels of re-epithelialization compared to application on ‘Day 0 only’—no significant differences were observed; ii. ‘Daily to Day 6’ application resulted in significantly increased levels of re-epithelialization compared to application on ‘Day 0 only’ over days 4 to 12 (p≤0.043); iii ‘Daily to Day 6’ application resulted in significantly increased levels of re-epithelialization compared to application ‘E4D’ over days 4 to 12 (p≤0.011).

All wounds in the study were visually assessed on a daily basis until day 8—and subsequently on alternate days until day 16—to establish their “healing” status. Each wound was scored as to whether it was displaying “neo-dermal tissue generation activity” within the central wound area. Scoring was undertaken independently by two independent observers and the average % of wounds displaying “neo-dermal tissue generation activity” was compared between treatment groups at each assessment point. The following points were apparent: None of the 10 wounds in the Vehicle Control group demonstrated initiation of wound healing over the 16-day study period. 2) All positive control treated wounds were responding (i.e., had initiated) by day 4 post-wounding (it should be noted that no initiation data were available for this historical control prior to day 4). This observed response was found to be statistically significant compared to Vehicle Control treatment from day 4 onwards (p=0.000, Fisher exact test). 3) Wounds in receipt of IMG-1T on ‘Day 0 only’ did not demonstrate any neo-dermal tissue formation during the 16-day study. 4) Wounds in receipt of IMG-1T applied ‘E4D’ demonstrated neo-dermal tissue formation from day 8 onwards, reaching an 80% response rate by day 16. This was significantly greater than both vehicle control wounds and wounds in receipt of IMG-1T applied ‘Day 0 only’—on days 12 to 16 (p≤0.033, Fisher exact test). 5) Wounds in receipt of IMG-1T applied ‘Daily to Day 6’ demonstrated neo-dermal tissue formation in 100% of wounds from day 4 onwards. This was comparable to the positive control treatment and significantly greater than wounds in receipt of IMG-1T applied ‘E4D’ on days 4 to 12 (p≤0.033, Fisher exact test).

This Example 2 examined the effect of (IMG-1T, 2 μg/mL in 0.5% HPMC), applied topically according to three dosing frequency regimens (Day 0 only, Every 4 Days [E4D] and Daily to Day 6), on the repair of full-thickness excisional skin wounds in the healing-impaired db/db diabetic mouse. The healing of wounds treated with IMG-1T (all regimens) was compared to Vehicle Control given E4D. Wound healing data generated during this study were compared to historical positive control data from wounds treated with a combination of recombinant human platelet-derived growth factor-BB (rh-PDGF-BB) and recombinant human Transforming Growth Factor-alpha (rh-TGF-alpha). Wound healing was assessed over a 16-day period in terms of (i) initiation of neo-dermal repair responses, and (ii) wound closure. Initiation of neo-dermal tissue formation was expressed as the number of wounds responding in each group at each time point. Wound closure was considered in both overall terms and in terms of its components wound contraction and wound re-epithelialization. Wound closure (contraction & re-epithelialization) was determined from digital photographs taken on post-wounding days 0, 4, 8, 12 & 16 post-wounding.

A single 100 μL dose of IMG-1T (2 μg/mL in 0.5% HPMC) applied on Day 0 was found to significantly increase the overall rate of wound closure compared to vehicle control treatment (applied E4D, CICA-IMA-01). On consideration of the components of wound healing, namely, wound contraction and wound re-epithelialization; this single dose resulted in significantly increased re-epithelialization, in tandem with reduced wound contraction. Application of IMG-1T (100 μL, 2 μg/mL) every four days (E4D) resulted in significantly increased wound closure compared to i) vehicle control treatment (E4D) and ii) IMG-1T applied on ‘Day 0 only’. This observed increase in wound closure, compared to the vehicle control group, consisted of both significantly increased wound contraction and increased wound re-epithelialization—suggesting a positive impact of IMG-1T on both components of wound closure. Dosing with IMG-1T ‘E4D’ also significantly increased the proportion of wounds demonstrating initiation of wound healing.

Application of IMG-1T (100 μL, 2 μg/mL) ‘Daily to day 6’ resulted in significantly increased wound closure compared to i) vehicle control treatment (E4D), ii) IMG-1T on ‘Day 0 only’, and iii) IMG-1T applied E4D.

### Example 3

As collagen is an essential part of wound healing, collagen production in neonatal Human Epidermal Keratinocytes, (HEKn) cells treated with IMG-1T comprising SEQ ID NO 1 was measured. Following 72 hours incubation with IMG-1T, HEKn cells were analyzed using a Human Pro-Collagen I alpha 1 ELISA Kit (AbCam). An increase in collagen deposition was viewed at concentrations as low as 1% IMG-1T (aqueous solution) (from 10.825 μg/mL collagen in the untreated to 15.825 μg/mL collagen in the cells treated with 1% IMG-1T), with a peak production of 20.7 μg/mL collagen at a concentration of 10% IMG-1T, a value almost twice that of untreated HEKn cells.

Next, it was determine if IMG-1T treatment of HEKn cells could cause an increase the production of elastin. Following 120 hours incubation with IMG-1T, HEKn cells were analyzed using a Human Elastin ELISA Kit (AbCam). Similar to collagen deposition, cells cultured in the presence of IMG-1T had increased levels of elastin, with an increase from 2 ng/mL to 2.3 ng/mL in 10% IMG-1T (aqueous solution) IMG-1T (aqueous solution) (corresponding to an increase of over 20% in elastin expression),

These results demonstrate that IMG-1T treatment of keratinocytes (in vitro) dramatically increases both collagen deposition and elastin production by keratinocytes.

### Example 4

IMG-1T can also be utilized for a variety of other skin and wound treatments, both cosmetically and medically. Split-thickness skin grafts are versatile adjuncts to wound closure in burns, trauma, reconstruction, and other large wounds. During a split-thickness skin graft, a surgeon removes a thin layer of skin from one part of a patient's body (donor site) and uses it to close the surgical site that needs to be covered (recipient site) on the patient. A split-thickness skin graft (STSG), by definition, refers to a graft that contains the epidermis and a portion of the dermis, which contrasts with a full-thickness skin graft (FTSG) which consists of the epidermis and entire dermis. Unlike flaps, skin grafts do not have their own blood supply and must rely on a well-vascularized wound bed for graft in-growth. Split-thickness skin grafts are obtainable from multiple sources (autograft, homograft, allograft, or xenograft), multiple anatomical locations, and in various thicknesses. Most commonly, STSG autografts are taken from the lateral thigh, as well as trunk, as these sites are both aesthetically hidden, as well as easy to harvest from due to their broad surfaces. Split-thickness skin grafts classify according to their thickness into thin STSGs (0.15 to 0.3 mm), intermediate STSGs (0.3 to 0.45 mm), and thick STSGs (0.45 to 0.6 mm). Because split-thickness skin graft donor sites retain portions of the dermis, including dermal appendages, the donor site can regrow new skin in 2 to 3 weeks. Thus, donor sites can be used more than once after appropriate healing has taken place, which makes STSGs versatile in burn surgery and large wounds where there are limited donor sites.

Though STSGs are a very common procedure, donor site morbidity can be a problem, especially regarding infection, which in turn increases the duration of post-procedure recovery and admission and increased cost of management. Though it is often believed that healing of the donor sites has a reported healing rate of <12 days, many studies report healing rates closer to 14-21 days. Within 24 hours many patients complain of exaggerated pain, itching, infection, dyschromia, hypopigmentation, hyper-pigmentation, and hypertrophic scars. With many patients having morbidity as late as 6 months post procedure. Hence improving the rate of healing and quality of wound repair are of utmost importance.

To test the ability of IMG-1T to treat a non-diabetic wound, split thickness wounds were generated on male Danish Landrace X Large White Crossbred pigs. The piglets were anesthetized by an isoflurane/oxygen mixture, which is delivered through a facemask. A 7×10 cm partial wound 400 mm deep was performed using Dermatome. Following the incision, the pigs received antibiotic (Marbocyl 10%) for 5 consecutive days. The animals were kept under anesthesia for the duration of the surgery and dosing. The study was designed to evaluate the effect of IMG-1T daily treatment on the healing of donor wounds. The pigs were exposed to 4 donor wounds per animal, with two wounds receiving IMG-1T (dose of 2 ug/mL in 1% HPMC gel vehicle) and two wounds receiving gel vehicle alone. The treated wounds (IMG-1T and vehicle only) were assessed daily and treated daily. The reduced areas of the wounds were evaluated every other day using ARANZ medical device. The IMG-1T treated wounds demonstrated a significant increase in wound area reduction as early as 3 days' post treatment, furthermore upon termination of the study histology was performed on the newly healed wounds and showed granularization depth was increased by over 25% in the IMG-1T treated animals.

### Example 5

As collagen is an essential part of wound healing, collagen production in neonatal Human Epidermal Keratinocytes, (HEKn), treated with a composition comprising SEQ ID No 2 was measured. Following 72 hours incubation, HEKn cells treated with a composition comprising SEQ ID No 2 were analyzed using a Human Pro-Collagen I alpha 1 ELISA Kit (AbCam). An increase in collagen deposition was viewed at both concentrations used in Example 3 (from 8.825 untreated to 18.85 and 17.125 ug/mL collagen with IMG-1 and 18.125 and 17.825 ug/mL collagen with IMG-2), with values almost twice that of untreated HEKn cells (See FIG. 7). These results demonstrate that SEQ ID NO 2 displays similar properties to SEQ ID NO 1 in regards to collagen deposition of keratinocytes and it's ability to be an affective therapeutic for the treatment of wounds.

### Embodiments and Aspects Thereof

Topical or incisional pharmaceutical compositions comprise an active ingredient, optionally in combination with a medication or drug or botanical (or combination thereof), and a pharmaceutically acceptable vehicle (or carrier). The pharmaceutically acceptable vehicle (or carrier) may comprise water, oil, alcohol, petrolatum, propylene glycol, glycerin, or a combination thereof mixed with one or more of a preservative, an emulsifier, an absorption promoter, and a fragrance. The combinations, ratio and grades selected thereof, to give the desired finished product viscosity/spreadability.

### First Embodiment

A first embodiment relates to a pharmaceutical composition for topical use comprises a therapeutically effective amount of a polypeptide of SEQ. ID NO. 1 or 2, or a derivative or analog thereof, and a pharmaceutically acceptable carrier.

A pharmaceutical composition disclosed herein may take the form of a solution, cream, ointment, paste, lotion, ointment, foam, spray, transdermal patch, or gel. Topical formulations are well characterized in the literature (See Benson, et al, Current Drug Deliv. 2019 June; 16(5): 444-460; Chang et al, AAPS J. 2015 Sep. 3; 17(6): 1522). Ointments, gels, creams, emulsions and foams are suitable vehicles for transdermal drug delivery, and IMG-1T may be formulated as an ointment, gel, cream, emulsions or foam, utilizing well-known and characterized pharmacological methods known in the art.

In one aspect of the first embodiment, a pharmaceutical composition for topical or incisional use comprises a therapeutically effective amount of a polypeptide according to SEQ. ID NO. 1 or 2 in an hydroxpropyl cellulose (HPMC) vehicle.

In one aspect, HPMC may be present in a concentration ranging from about 0.25% w/v to about 2.5% w/v. HPMC is biocompatible, has hydration and gel forming properties and has global regulatory acceptance to be used in the preparation of various pharmaceutical formulations. HPMC is usually used to extend the release time of drugs.

Cellulose derivatives-based hydrogels, such as hydroxypropyl methylcellulose (aka, hypromellose or HPMC), carboxymethyl cellulose (CMC) or a salt thereof (e.g., carboxymethyl cellulose sodium), hydroxyethyl methylcellulose (NEMC), are all useful as transdermal drug-delivery systems due to their excellent properties, including: (i) their simple application, (ii) reduction of the systemic side effects, (iii) avoidance of the liver first-pass effect, and (iv) capacity to provide an improved feeling for the skin in comparison with other conventional unguents and patches. In the case of hydrogels based on cellulose derivatives, different studies have been reported related to the transdermal delivery of different drugs. (See Ciolacu, et al, Cellulose-Based Hydrogels as Sustained Drug-Delivery Systems, Materials 2020, 13, 5270; and Cinie et al, U.S. Pat. No. 5,457,093; Lachman et al. The Theory and Practice of Industrial Pharmacy, 1987, Chapter 18 (“Semisolids”), pp. 534-563).

In one aspect of the first embodiment, the pharmaceutical composition may be applied topically or incisionally, as the circumstance may require. In yet another aspect, the pharmaceutical composition may be in the form of a solution, a cream, an ointment, a paste, a lotion, an ointment, a foam, a spray, a transdermal patch, or a gel.

One aspect of the first embodiment relates to a pharmaceutical composition comprises a therapeutically effective amount of a polypeptide of SEQ. ID NO. 1 or 2, or a derivative or analog thereof, and a pharmaceutically acceptable carrier comprising a cellulose derivative-based hydrogel.

In one aspect of the first embodiment, the pharmaceutical composition comprises an amount of SEQ. ID NO. 1 or 2, or a derivative or analog thereof, ranges from about μg/mL up to about 10 μg/mL and all values in between, including, for example about μg/mL, about 1 μg/mL, about 1.5 μg/mL, about 2 μg/mL, about 2.5 μg/mL, about 3 μg/mL, about 3.5 μg/mL, about 4 μg/mL, about 4.5 μg/mL, about 5 μg/mL, about 5.5 μg/mL, about 6 μg/mL, about 6.5 μg/mL, about 7 μg/mL, about 7.5 μg/mL, about 8, μg/mL about 8.5 μg/mL, about 9 μg/mL, and about 9.5 μg/mL.

Yet another aspect of the first embodiment relates to a pharmaceutical composition comprising about 0.1 μg/mL to about 10 μg/mL of a polypeptide of SEQ. ID NO. 1 or 2, or a derivative or analog thereof, and a pharmaceutically acceptable carrier.

Yet another aspect of the first embodiment relates to a pharmaceutical composition comprising about 0.1 μg/mL to about 10 μg/mL of a polypeptide of SEQ. ID NO. 1 or 2 and a pharmaceutically acceptable carrier.

Yet another aspect of the first embodiment relates to a topical pharmaceutical composition comprising about 2 μg/mL of a polypeptide of SEQ. ID NO. 1 or 2 or a derivative thereof and a pharmaceutically acceptable carrier.

Yet another aspect of the first embodiment relates to a topical pharmaceutical composition comprising about 2 μg/mL of a polypeptide of SEQ. ID NO. 1 or 2 and a pharmaceutically acceptable carrier.

In another aspect of the first embodiment, the pharmaceutical composition comprises an amount of SEQ. ID NO. 1 or 2, or a derivative or analog thereof, that ranges from about 0.01% w/v to about 10% w/v, and all values in between, including, for example, about 0.02% w/v, about 0.03% w/v, about 0.04% w/v, about 0.05% w/v, about w/v, about 0.07% w/v, about 0.08% w/v, about 0.09% w/v, about 0.1% w/v, about w/v, about 0.2% w/v, about 0.25% w/v, about 0.3% w/v, about 0.35% w/v, about w/v, about 0.5% w/v, about 1.0% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, 7 about % w/v, about 7.5% w/v, about 8% w/v, about 8.5% w/v, about 9% w/v, and about 9.5% w/v.

Yet another aspect of the first embodiment relates to a topical pharmaceutical composition comprising from of about 0.01% w/v to about 0.1% w/v of a polypeptide of SEQ. ID NO. 1 or 2, or a derivative or analog thereof, and a pharmaceutically acceptable carrier.

Yet another aspect of the first embodiment relates to a topical pharmaceutical composition comprising about 0.02% w/v of a polypeptide of SEQ. ID NO. 1 or 2, a derivative or analog thereof, and a pharmaceutically acceptable carrier.

Yet another aspect of the first embodiment relates to a topical pharmaceutical composition comprising from of about 0.01% w/v to about 0.1% w/v of a polypeptide of SEQ. ID NO. 1 or 2 and a pharmaceutically acceptable carrier.

Yet another aspect of the first embodiment relates to a topical pharmaceutical composition comprising about 0.02% w/v of a polypeptide of SEQ. ID NO. 1 or 2 and a pharmaceutically acceptable carrier.

### Second Embodiment

A second embodiment relates to a pharmaceutical composition comprising a means for promoting wound healing and a pharmaceutically acceptable carrier.

A first aspect of the second embodiment relates to a pharmaceutical composition comprising a means for promoting wound healing and a pharmaceutically acceptable carrier wherein the means for promoting would healing is a polypeptide of SEQ ID No. 1 or 2, or a derivative or analog thereof.

A second aspect of the second embodiment relates to a pharmaceutical composition comprising a means for promoting wound healing and a pharmaceutically acceptable carrier wherein the means for promoting would healing is a polypeptide of SEQ ID No. 1 or 2.

The pharmaceutical compositions disclosed herein exhibit several unexpected properties, including, for example, wound healing, promotion of collagen and/or elastin production in a wounded tissue, increased wound healing, and improvements in wound contraction and/or wound re-epithelialization.

### Third Embodiment

Accordingly, a third embodiment relates to a method for the treatment of a wound in a mammal (e.g., a human, a human patient, etc.), which comprises applying a pharmaceutical composition comprising: a therapeutically effective amount of a polypeptide of SEQ ID No. 1 or 2, or a derivative or analog thereof, and a pharmaceutically acceptable carrier to the wound of the mammal (e.g., a human, a human patient, etc.).

One will appreciate that a polypeptide of SEQ ID No. 1 or 2, or a derivative or analog thereof, shows efficacy with respect to the treatment of a chronic wound, which includes, but is not limited to a skin ulcer, an infectious wound, an ischemic wound, a surgical wound, a skin wound from radiation poisoning, or a combination thereof.

Accordingly, a first aspect of the third embodiment relates to a method for the treatment of a wound in a mammal (e.g., a human, a human patient, etc.), which comprises applying a pharmaceutical composition comprising: a therapeutically effective amount of a polypeptide of SEQ ID No. 1 or 2, or a derivative or analog thereof, and a pharmaceutically acceptable carrier to the wound of the mammal (e.g., a human, a human patient, etc.); wherein the wound comprises a skin ulcer, an infectious wound, an ischemic wound, a surgical wound, a skin wound from radiation poisoning, or a combination thereof.

Certain skin ulcers may be categorized as a diabetic foot ulcer. One will appreciate that there are several types of diabetic foot ulcers, including (i) a neuropathic ulcer (which may occur where there is peripheral diabetic neuropathy, but no ischemia caused by peripheral artery disease); (ii) an ischemic ulcer (which may occur where there is peripheral artery disease present without the involvement of diabetic peripheral neuropathy); and (iii) a neuroischemic ulcer (which may occur where the mammal (e.g., human) has both peripheral neuropathy and ischemia resulting from peripheral artery disease).

Accordingly, one aspect of the third embodiment relates to a method for the treatment of a wound in a mammal (e.g., a human, a human patient, etc.), which comprises applying a pharmaceutical composition comprising: a therapeutically effective amount of a polypeptide of SEQ ID No. 1 or 2, or a derivative or analog thereof, and a pharmaceutically acceptable carrier to the wound of the mammal (e.g., a human, a human patient, etc.); wherein the wound comprises a skin ulcer, which comprises a neuropathic ulcer an ischemic ulcer a neuroischemic ulcer, or a combination thereof.

Based on results presented herein, as well as U.S. Provisional Patent Application No. 63/122,746, one may appreciate that applying a pharmaceutical composition of the first or second embodiment to a mammal (e.g., a human) results in several unexpected properties, including, for example, wound healing, promotion of collagen and/or elastin production in a wounded tissue, increased wound healing, and improvements in wound contraction and/or wound re-epithelialization.

For instance, applying a therapeutically effective amount of a polypeptide of SEQ. ID NO. 1 or 2 (viz., IMG-1T) results in increased collagen production, as measured in an in vitro assay using neonatal Human Epidermal Keratinocytes (“HEKn”). Incubating HEKn cells with a concentration of IMG-1T (about 1% w/v to about 10% w/v) resulted in a measurable increase in collagen when compared to untreated HEKn cells. Following 72 hours incubation with IMG-1T, HEKn cells were analyzed using a Human Pro-Collagen I alpha 1 ELISA Kit (AbCam). After 72 hours, untreated cells exhibited a collagen content of about 10.8 μg/mL. An increase in collagen deposition was observed at about 1% w/v IMG-1T (about 15.8 μg/mL) and at about 10% w/v IMG-1T (about 20.7 μg/mL). Accordingly, the observations showed that applying IMG-1T resulted in an increased collagen content, including, for example, a collagen production increase of about 45% to about 100%, relative to untreated control.

Accordingly, one aspect of the third embodiment relates to a method for the treatment of a wound in a mammal (e.g., a human, a human patient, etc.), which comprises applying a pharmaceutical composition comprising: a therapeutically effective amount of a polypeptide of SEQ ID No. 1 or 2, or a derivative or analog thereof, and a pharmaceutically acceptable carrier to the wound of the mammal (e.g., a human, a human patient, etc.), wherein the applying results in an increased collagen production, which may range from about 45% to about 100%, relative to untreated control.

Following 120 hours incubation with IMG-1T, HEKn cells were analyzed using a Human Elastin ELISA Kit (AbCam). Similar to collagen deposition, cells cultured in the presence of IMG-1T had increased levels of elastin, with an increase from 2 ng/mL to 2.3 ng/mL in 10% Active agent (corresponding to an increase of −11%) and to 2.6 ng/mL at 20% Active agent (corresponding to an increase of over 20% in elastin expression).

Accordingly, one aspect of the third embodiment relates to a method for the treatment of a wound in a mammal (e.g., a human, a human patient, etc.), which comprises applying a pharmaceutical composition comprising: a therapeutically effective amount of a polypeptide of SEQ ID No. 1 or 2, or a derivative or analog thereof, and a pharmaceutically acceptable carrier to the wound of the mammal (e.g., a human, a human patient, etc.), wherein the applying results in an increased elastin production, which may range from about 10% to about 20%, relative to untreated control.

The HEKn assay results demonstrate that IMG-1T may not improve the proliferation of keratinocytes, but IMG-1T substantially increases the number of CD133 keratinocyte progenitor cells in the cell population, and also increases both collagen deposition and elastin production of keratinocytes.

Further, applying a therapeutically effective amount of a polypeptide of SEQ. ID NO. 1 (viz., IMG-1T) results in increased wound healing, as measured by a study that investigated the percentage of wound area remaining, see Table 2 results (see also FIG. 1). For instance, applying IMG-1T (2 μg/mL) to a wound every fourth day resulted in a substantial reduction in wound area remaining, relative to untreated control. With reference to the Table 2 data (see also FIG. 1, as well as Table 6), one may appreciate that applying IMG-1T at about 2 μg/mL resulted in about 1.8% of wound area remaining after 24 days, applying IMG-1T at about 4 μg/mL resulted in about 5.9% of wound area remaining after 24 days, while the untreated control animals resulted in about 34% of wound area remaining after 24 days.

Accordingly, one aspect of the third embodiment relates to a method for the treatment of a wound in a mammal (e.g., a human, a human patient, etc.), which comprises applying a pharmaceutical composition comprising: a therapeutically effective amount of a polypeptide of SEQ ID No. 1 or 2, or a derivative or analog thereof, and a pharmaceutically acceptable carrier to the wound of the mammal (e.g., a human, a human patient, etc.), wherein the applying results in an reduced wound area remaining of about 6% or lower after 24 days, relative to untreated control, including about 5% or lower, about 4% or lower, about 3% or lower, and about 2% or lower.

The Table 6 data shows that applying IMG-1T daily to day six and then every four days thereafter, showed a substantial improvement in the percentage wound area remaining. In view of the foregoing, one may appreciate that an administration schedule may be based on the observations of an attending physician and that a pharmaceutical composition may be used as directed. In certain instances, it may be convenient to prescribe a certain dosage amount of IMG-1T (e.g., 2 μg/mL) on an administration schedule of twice daily, daily, every other day, every third day, every fourth day, and the like.

Accordingly, one aspect of the third embodiment relates to a method for the treatment of a wound in a mammal (e.g., a human, a human patient, etc.), which comprises applying daily a pharmaceutical composition comprising: a therapeutically effective amount of a polypeptide of SEQ ID No. 1 or 2, or a derivative or analog thereof, and a pharmaceutically acceptable carrier to the wound of the mammal (e.g., a human, a human patient, etc.).

Further, another aspect of the third embodiment relates to a method for the treatment of a wound in a mammal (e.g., a human, a human patient, etc.), which comprises applying daily a pharmaceutical composition comprising about 1 μg/mL to about 10 μg/mL (e.g., 2 μg/mL) of a polypeptide of SEQ ID No. 1 or 2, or a derivative or analog thereof, and a pharmaceutically acceptable carrier to the wound of the mammal (e.g., a human, a human patient, etc.).

Moreover, another aspect of the third embodiment relates to a method for the treatment of a wound in a mammal (e.g., a human, a human patient, etc.), which comprises applying daily a pharmaceutical composition comprising about 1 μg/mL to about 10 μg/mL (e.g., 2 μg/mL) of a polypeptide of SEQ ID No. 1 or 2 and a pharmaceutically acceptable carrier to the wound of the mammal (e.g., a human, a human patient, etc.).

Another aspect of the third embodiment relates to a method for the treatment of a wound in a mammal (e.g., a human, a human patient, etc.), which comprises applying daily a pharmaceutical composition comprising about 1 μg/mL to about 10 μg/mL (e.g., 2 μg/mL) of a polypeptide of SEQ ID No. 1 or 2 and a pharmaceutically acceptable carrier to the wound of the mammal (e.g., a human, a human patient, etc.), wherein the pharmaceutical composition is in the form of a solution, a cream, an ointment, a paste, a lotion, an ointment, a foam, a spray, a transdermal patch, or a gel.

Additionally, applying a therapeutically effective amount of a polypeptide of SEQ. ID NO. 1 or 2 (viz., IMG-1T) results in increased wound healing, as measured by a study that investigated the percentage wound contraction, see Table 3 results (see also FIG. 2 and Table 7). For instance, applying IMG-1T (2 μg/mL) to a wound every fourth day resulted in a substantial would contraction, relative to untreated control. With reference to the Table 3 data (see also FIG. 2), one may appreciate that applying IMG-1T at about 2 μg/mL resulted in about 68.9% of wound contraction after 24 days, applying IMG-1T at about 4 μg/mL resulted in about 64.7% of wound contraction after 24 days, while the untreated control animals resulted in about 43.4% of wound area remaining after 24 days.

Accordingly, one aspect of the third embodiment relates to a method for the treatment of a wound in a mammal (e.g., a human, a human patient, etc.), which comprises applying a pharmaceutical composition comprising: a therapeutically effective amount of a polypeptide of SEQ ID No. 1 or 2, or a derivative or analog thereof, and a pharmaceutically acceptable carrier to the wound of the mammal (e.g., a human, a human patient, etc.), wherein the applying results in an percentage wound contraction of about 40% or more after 24 days, including about 65% or more.

Additionally, applying a therapeutically effective amount of a polypeptide of SEQ. ID NO. 1 or 2 (viz., IMG-1T) results in increased wound healing, as measured by a study that investigated the percentage improvements in wound re-epithelialization, see Table 4 results (see also FIG. 3 and Table 8). For instance, applying IMG-1T (2 μg/mL) to a wound every fourth day resulted in a substantial an improvement in wound re-epithelialization, relative to untreated control. With reference to the Table 4 data (see also FIG. 3), one may appreciate that applying IMG-1T at about 2 μg/mL resulted in about 29.3% of wound area remaining after 24 days, applying IMG-1T at about 4 μg/mL resulted in about 29.4% of wound area remaining after 24 days, while the untreated control animals resulted in about 22.5% of wound area remaining after 24 days. And the data presented in Table 8 demonstrates that applying 2 μg/mL of IMG-1T daily (to day 6) and every fourth day (E4D) for the remainder of the treatment period showed a percent re-epithelialization of about 51.6% after 24 days.

Accordingly, one aspect of the third embodiment relates to a method for the treatment of a wound in a mammal (e.g., a human, a human patient, etc.), which comprises applying a pharmaceutical composition comprising: a therapeutically effective amount of a polypeptide of SEQ ID No. 1 or 2, or a derivative or analog thereof, and a pharmaceutically acceptable carrier to the wound of the mammal (e.g., a human, a human patient, etc.), wherein the applying results in an improved percentage of wound re-epithelization of at least 30% after 24 days.

One will appreciate that additional advantages of the pharmaceutical composition disclosed herein may be gleaned from the results presented herein.

### ADDITIONAL FEATURES

Feature 1. A pharmaceutical composition for promoting wound healing, comprising: a therapeutically effective amount of a polypeptide of SEQ ID No. 1 or 2, or a derivative or analog thereof, and pharmaceutically acceptable carrier.

Feature 2. The pharmaceutical composition of feature 1 in the form of a solution, a cream, an ointment, a paste, a lotion, an ointment, a foam, a spray, a transdermal patch, or a gel.

Feature 3. The pharmaceutical composition of any one of the preceding features, wherein the therapeutically effective amount of the polypeptide of SEQ ID No. 1 or 2, or a derivative or analog thereof ranges from about 0.1 μg/mL to about 10 μg/mL.

Feature 4. The pharmaceutical composition of any one of the preceding features, wherein the therapeutically effective amount of the polypeptide of SEQ ID No. 1 or 2 ranges from about 0.1 μg/mL to about 10 μg/mL.

Feature 5. The pharmaceutical composition of any one of the preceding features comprising about 2 μg/mL of the polypeptide of SEQ ID No. 1 or 2.

Feature 6. A pharmaceutical composition comprising a means for promoting wound healing and a pharmaceutically acceptable carrier.

Feature 7. The pharmaceutical composition of feature 6, wherein the means for promoting would healing is a polypeptide of SEQ ID No. 1 or 2 or a derivative or analog thereof.

Feature 8. The pharmaceutical composition of feature 6, wherein the means for promoting would healing is a polypeptide of SEQ ID No. 1 or 2.

Feature 9. A method for the treatment of a wound in a mammal, which comprises applying a pharmaceutical composition comprising: a therapeutically effective amount of a polypeptide of SEQ ID No. 1 of 2, or a derivative or analog thereof, and pharmaceutically acceptable carrier to the wound of the mammal.

Feature 10. The method of feature 9, wherein the wound comprises a skin ulcer, an infectious wound, an ischemic wound, a surgical wound, a skin wound from radiation poisoning, or a combination thereof.

Feature 11. The method of any one of features 9-10, wherein the wound is a skin ulcer comprising a neuropathic ulcer an ischemic ulcer, a neuroischemic ulcer, or a combination thereof.

Feature 12. The method of any one of features 9-11, wherein the applying increases collagen production when compared to an untreated control.

Feature 13. The method of any one of features 9-12, wherein the applying increases elastin production when compared to an untreated control.

Feature 14. The method of any one of features 9-13, wherein the applying increases collagen and/or elastin production when compared to an untreated control.

Feature 15. The method of any one of features 9-14, which further comprises applying to the wound a therapeutically effective amount of a human platelet-derived growth factor-BB (rh-PDGF-BB), a human Transforming Growth Factor-alpha (rh-TGF-alpha), or a combination thereof.

Feature 16. The pharmaceutical composition of feature 1, wherein the peptide has at least 95% sequence identity to SEQ ID NO. 1 or 2, or a derivative or analog thereof, optionally at least 98% sequence identity to SEQ ID NO. 1 or 2, or a derivative or analog thereof, further optionally at least 99% sequence identity to SEQ ID NO. 1 or 2, or a derivative or analog thereof.

Feature 17. The pharmaceutical composition of any preceding feature, wherein the pharmaceutical carrier or vehicle is selected from a modified cellulose (such as hydroxypropyl cellulose (HPMC), carboxymethylcellulose (CMC), or hydroxyethylmethyl cellulose (NEMC)), hypromellose, physiological buffer (such as phosphate buffered saline), gelatin, or hydrogel.

Feature 18. The pharmaceutical composition of any preceding feature, wherein the pharmaceutical vehicle is present at a concentration of not more than about 10% w/w, optionally wherein the pharmaceutical vehicle is present at a concentration of not more than about 5% w/w.

Feature 19. The pharmaceutical composition of feature 17, wherein the pharmaceutical vehicle is HPMC, optionally wherein the HPMC is present in an amount of from about 0.5% w/w to about 5% w/w.

Feature 20. The pharmaceutical composition of any of preceding feature, wherein treatment comprises promotion of wound healing, and/or promotion of collagen and/or elastin production in a wounded tissue, and/or improvements in wound contraction, and/or wound re-epithelialization

It will be clear to a person skilled in the art that features described in relation to any of the embodiments described above can be applicable interchangeably between the different embodiments. The embodiments described above are examples to illustrate various features of the compositions and methods disclosed herein.

Throughout the description and claims of this specification, the words “comprise” and “contains” and variations of them mean “including but not limited to”, and they are not intended to (and do not) exclude other moieties, additives, components, or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, characteristics, compounds, chemical moieties or groups described in conjunction with a particular aspect, embodiment or example are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The compositions and methods disclosed herein are not restricted to the details of any foregoing embodiments.

The reader's attention is directed to all papers and documents which are filed or referenced concurrently with, or previous to, this specification in connection with this application and any which are open to public inspection that are referenced with this specification, and the contents of all such papers and documents are incorporated herein by reference. For instance, the subject matter of U.S. provisional patent application No. 63/122,746, filed on Dec. 8, 2020, is incorporated by reference in its entirety.

1. A pharmaceutical composition for promoting wound healing, comprising: a therapeutically effective amount of a polypeptide of SEQ ID NO. 1 or 2, or a derivative or analog thereof, and pharmaceutically acceptable carrier.

2. The pharmaceutical composition of claim 1 in the form of a solution, a cream, an ointment, a paste, a lotion, an ointment, a foam, a spray, a transdermal patch, or a gel.

3. The pharmaceutical composition of claim 1, wherein the therapeutically effective amount of the polypeptide of SEQ ID NO. 1 or 2, or a derivative or analog thereof, ranges from about 0.1 μg/mL to about 10 μg/mL.

4. The pharmaceutical composition of any preceding claim, wherein the pharmaceutical carrier is selected from a modified cellulose (such as hydroxypropyl cellulose (HPMC), carboxymethylcellulose (CMC), or hydroxyethylmethyl cellulose (NEMC)), hypromellose, physiological buffer, gelatin, hydrogel, oil, alcohol, petrolatum, propylene glycol, glycerin, or a combination thereof.

5. The pharmaceutical composition of claim 1 comprising about 2 μg/mL of the polypeptide of SEQ ID NO. 1 or 2, or a derivative or analog thereof.

6. A pharmaceutical composition comprising a means for promoting wound healing and a pharmaceutically acceptable carrier.

7. The pharmaceutical composition of claim 6, wherein the means for promoting would healing is a polypeptide of SEQ ID NO. 1 or 2, or a derivative or analog thereof.

8. The pharmaceutical composition of claim 6, wherein the means for promoting would healing is a polypeptide of SEQ ID NO. 1 or 2, or a derivative or analog thereof.

9. A method for the treatment of a wound in a mammal, which comprises applying a pharmaceutical composition comprising: a therapeutically effective amount of a polypeptide of SEQ ID NO. 1 or 2, or a derivative or analog thereof, and pharmaceutically acceptable carrier to the wound of the mammal.

10. The method of claim 9, wherein the wound comprises a skin ulcer, an infectious wound, an ischemic wound, a surgical wound, a skin wound from radiation poisoning, or a combination thereof.

11. The method of claim 9, wherein the wound is a skin ulcer comprising a neuropathic ulcer an ischemic ulcer, a neuroischemic ulcer, or a combination thereof.

12. The method of claim 9, wherein the applying increases collagen production when compared to an untreated control.

13. The method of claim 9, wherein the applying increases elastin production when compared to an untreated control.

14. The method of claim 9, wherein the applying increases collagen and/or elastin production when compared to an untreated control.

15. The method of claim 9, which further comprises applying to the wound a therapeutically effective amount of a human platelet-derived growth factor-BB (rh-PDGF-BB), a human Transforming Growth Factor-alpha (rh-TGF-alpha), or a combination thereof.

16. A pharmaceutical composition for topical administration comprising a peptide having at least 90% sequence identity to SEQ ID NO. 1 or 2, or a derivative or analog thereof, and a pharmaceutical vehicle.

17. A peptide having at least 90% sequence identity to SEQ ID NO. 1 or 2, or a derivative or analog thereof, for topical use in promotion of wound healing.

18. The pharmaceutical composition of any preceding claim, wherein the pharmaceutical carrier is HPMC, optionally wherein the HPMC is present in an amount of from about 0.5% w/w to about 10% w/w.
